(19) United States
(12) Patent Application Publication
(10) Pub. No.: US 2025/0388840 A1
(43) Pub. Date: Dec. 25, 2025

(54) SUSTAINED PERFUSION DEVICE IN MODULAR MICROFLUIDIC DEVICES

(71) Applicant: Ohio State Innovation Foundation, Columbus, OH (US)

(72) Inventors: Shashwat Agarwal, Columbus, OH (US); Brendan Fuller, Columbus, OH (US); Jacob Holter, Columbus, OH (US); Travis Jones, Columbus, OH (US); Jonathan Song, Columbus, OH (US)

(21) Appl. No.: 19/245,962

(22) Filed: Jun. 23, 2025

Related U.S. Application Data
(60) Provisional application No. 63/662,978, filed on Jun. 21, 2024.

(51) Int. Cl. C12M 3/00; C12M 1/00; C12M 1/12; C12M 1/34; C12M 3/06

(52) U.S. Cl. CPC C12M 21/08; C12M 23/16; C12M 25/04; C12M 29/10; C12M 41/40

(57) ABSTRACT

A system may include a liquid hopper including a hopper wall defining an internal volume, an inlet coupled to the hopper wall, a funnel coupled to the hopper wall and defining an outlet in fluid communication with the inlet, a hydraulic resistor mount coupled to the hopper wall between the inlet and the outlet, and a hydraulic resistor coupled to the membrane mount. A system may include a microfluidic device comprising an extracellular matrix (ECM) derived hydrogel, and defining a lumen therethrough fluidly coupled to the outlet of the liquid hopper, wherein when liquid flows from the outlet and subsequently into the lumen of the microfluidic device, the liquid is permitted to perfuse at least one of: i) the lumen and ii) a wall of the microfluidic device.

### CROSS REFERENCE TO RELATED APPLICATIONS

This application claims the benefit of U.S. Provisional Patent Application No. 63/662,978 filed on Jun. 21, 2024, the entire contents of which are incorporated herein by reference.

### BACKGROUND

The present disclosure relates generally to medical devices and more particularly the present disclosure relates to perfusion devices and synthetic biological microfluidic systems. In some implementations, this disclosure relates to microfluidic devices such as micro-scale systems, and microfluidic devices used in the food, water, cosmetics, and/or life science industries.

### SUMMARY

In some aspects, the disclosure relates to a system, including: a liquid hopper including a hopper wall defining an internal volume, an inlet coupled to the hopper wall, a funnel coupled to the hopper wall and defining an outlet in fluid communication with the inlet, a hydraulic resistor mount coupled to the hopper wall between the inlet and the outlet, and a hydraulic resistor coupled to the membrane mount; and a microfluidic device including an extracellular matrix (ECM) derived hydrogel, and defining a lumen therethrough fluidly coupled to the outlet of the liquid hopper; wherein when liquid flows from the outlet and subsequently into the lumen of the microfluidic device, the liquid is permitted to perfuse at least one of: i) the lumen and ii) a wall of the microfluidic device.

In some aspects, the disclosure relates to a system, wherein the microfluidic device is a first microfluidic device and the ECM derived hydrogel is a first ECM derived hydrogel, and further including a second microfluidic device fluidly coupled to the first microfluidic device, wherein the second microfluidic device includes a second ECM derived hydrogel different than the first ECM derived hydrogel.

In some aspects, the disclosure relates to a system, further including a microfluidic device container housing the first microfluidic device and the second microfluidic device, wherein the microfluidic device container includes a first pressure port associated with the first microfluidic device, and a second pressure port associated with the second microfluidic device.

In some aspects, the disclosure relates to a system, wherein the microfluidic device container includes a lumen pressure port.

In some aspects, the disclosure relates to a system, wherein the outlet includes a needle hub.

In some aspects, the disclosure relates to a system, wherein the outlet includes a luer lock or a slip tip.

In some aspects, the disclosure relates to a system, wherein when the liquid hopper is coupled to the microfluidic device, and wherein the hopper wall is situated gravitationally higher than the funnel and the microfluidic device.

In some aspects, the disclosure relates to a system, wherein the hydraulic resistor mount includes a shoulder between the hopper wall and the funnel.

In some aspects, the disclosure relates to a system, wherein the liquid hopper is configured to contain a liquid, a polymer, a cellular suspension, a solid suspension, a soft solid suspension, a liquid solvent with solutes, nanoparticles, or DNA structures.

In some aspects, the disclosure relates to a system, wherein the liquid hopper further includes a lid selectively coupled to the hopper wall and configured to reduce evaporation.

In some aspects, the disclosure relates to a system, wherein the hopper wall is cylindrical.

In some aspects, the disclosure relates to a system, wherein a diameter of the hopper wall is between 1 mm and 130 mm.

In some aspects, the disclosure relates to a system, wherein the hydraulic resistor is a semipermeable membrane.

In some aspects, the disclosure relates to a system, wherein the semipermeable membrane is a disc or a wafer.

In some aspects, the disclosure relates to a system, wherein the hydraulic resistor includes a porous material or a microchannel.

In some aspects, the disclosure relates to a system, wherein a hydraulic diameter of the hydraulic resistor is between 1 μm and 500 um.

In some aspects, the disclosure relates to a system, wherein a length of the hydraulic resistor is between 10 μm and 100 mm.

In some aspects, the disclosure relates to a system, wherein the hydraulic resistor defines a spiral shape.

In some aspects, the disclosure relates to a system, wherein the microfluidic device includes a synthetic microvessel.

In some aspects, the disclosure relates to a system, wherein a positive hydrostatic pressure differential across the hydraulic resistor is established based on a gravitational head of the liquid within the internal volume.

In some aspects, the disclosure relates to an automated liquid handling system for high throughput, the automatic liquid handling system including:

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a depiction of transvascular and interstitial drug transport by either diffusion or convection, according to some implementations;

FIG. 2A is a flow diagram of fabricating a PDMS-based microfluidic device, such as a micro-engineered and perfusable synthetic blood vessel, according to some implementations;

FIG. 2B is a confocal microscopy image of a fibrillar ECM in the microdevice and embedded with an open lumen structure ˜250-300 μm in diameter, according to some implementations;

FIG. 2C is a confocal projection of intact human umbilical vein endothelial cell (HUVEC) microvessel stained for VE-cadherin junctions (yellow) and DAPI for nuclei (blue), according to some implementations;

FIG. 2D is a three dimensional (3D) confocal microscopy image of a vessel lumen stained for actin (phalloidin, red) and nuclei (DAPI, blue), according to some implementations;

FIG. 2E is a depiction of fluorescence intensity of the tracer molecule (10 kDa FITC conjugated dextran) at an initial time, used to, for example, calculate apparent vascular permeability over time, according to some implementations;

FIG. 2F is a depiction of the fluorescence intensity of the tracer molecule (10 kDa FITC conjugated dextran) of FIG. 2E after 300 seconds, used to, for example, calculate apparent vascular permeability over time, according to some implementations;

FIG. 2G is a bar chart depicting test data showing recombinant CXCL12−α (100 ng/ml) increased vessel permeability in Col only and Col+HA ECM. **, p<0.01. n=4 per condition, according to some implementations;

FIG. 2H is a side perspective view of a liquid hopper having an open top and configured to facilitate pumpless microfluidic control of a perfusable microvessel such as the perfusable synthetic blood vessel of FIG. 2A, according to some implementations;

FIG. 2I is a schematic diagram of the head and head losses of the liquid hopper coupled to a perfusable microvessel, according to some implementations;

FIG. 3A is a top view of a liquid hopper coupled to a microvessel having a lumen defined through a first ECM region and a second ECM region distinct from the first ECM region, where the first ECM region and the second ECM region are in a series arrangement, according to some implementations;

FIG. 3B is an image of the perfusable microvessel of FIG. 3A showing two distinct ECM regions embedded with different fluorescent beads, where the scale bar is 500 μm, according to some implementations;

FIG. 4A is a depiction of a fluid hopper coupled to a perfusable microvessel showing different microvessel perfusion states due to transmural coupling of intravascular, transvascular, and interstitial fluid flow, where the port for the ECM 1 region and the port for the ECM 2 region are both sealed with a biocompatible tape or a VALAP wax to block transvascular flow (TVF), according to some implementations;

FIG. 4B is a depiction of the fluid hopper coupled to the perfusable microvessel of FIG. 4A, showing outward TVF becoming interstitial flow in ECM 1 and ECM 2 due to equilibrated intravascular fluid pressure (IVP) being greater than the interstitial fluid pressure (IFP) in the ECM 1 and ECM 2 regions (IFP 1 and IFP 2 respectively), where the port for the ECM 1 region and the port for the ECM 2 region are not sealed, according to some implementations;

FIG. 4C is a depiction of the fluid hopper coupled to the perfusable microvessel of FIG. 4A, showing outward TVF adjacent to ECM 1 (due to IVP>IFP 1) and inward TVF adjacent to ECM 2 (due to IVP<IFP 2), where the port for the ECM 2 region is subject to an external head pressure, according to some implementations; and

FIG. 5 is an image of engineered blood and lymphatic vessel within hydrogel of microtissue-engineered model, with solid arrows indicating intravascular perfusion and dashed arrows indicating interstitial flow, and the scale bar is 300 μm, according to some implementations.

### DETAILED DESCRIPTION

Before turning to the figures, which illustrate the exemplary implementations in detail, it should be understood that the present application is not limited to the details or methodology set forth in the description or illustrated in the figures. It should also be understood that the terminology is for the purpose of description only. Like reference numerals in the figures may represent and refer to the same or similar element, feature, or function.

Long-term perfusion in microfluidic systems can be implemented in a range of applications such as pharmaceuticals, cosmetics, food and water quality, thermal cooling applications, etc. For example, in the case of medicine, in the past 25 years, only 9.6% of drugs that entered Phase I clinical trials have secured United States Food and Drug Administration (FDA) approval. It is estimated that $2.8 billion is a financial cost to bring a drug to market. Moreover, the drug approval process can be lengthy, typically involving more than 10 years. A lack of accurate drug screening platforms to estimate drug efficacy and toxicity contributes to the high financial costs and often protracted time scales associated with obtaining drug approvals. The FDA's Phase III clinical trials are the most expensive in the drug approval pipeline. About 50% of drugs fail to secure approval at this stage. By employing microfluidic systems complemented with preclinical in vivo data, comprehensive drug screening will help to stratify earlier in the clinical trial pipeline candidate drugs from those that would ultimately fail thereby reducing cost and time for the pharmaceutical industry.

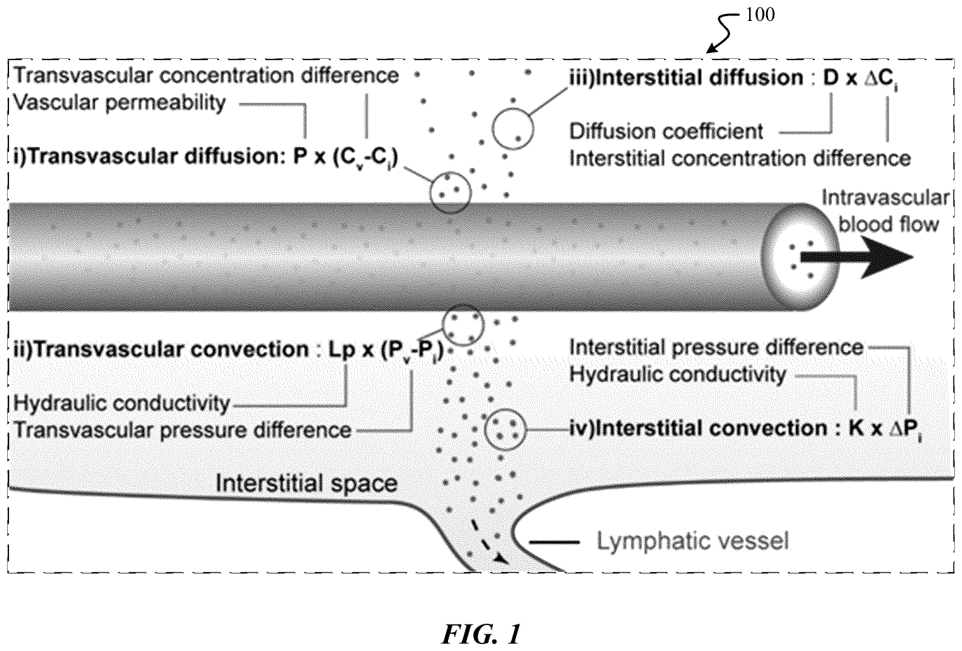

With respect to cancer and anti-cancer therapies, one longstanding objective is to increase the amount of a systemically administered drug that reaches local cancer cells in a tumor to improve efficacy and clinical outcomes for patients. The systems and methods described herein provide technical solutions to these and other longstanding technical problems that enable users to investigate both blood and lymphatic vascular systems in tissue engineered systems with extracellular matrix (ECM) composition, mechanics, and interstitial fluid flow that recapitulate in vivo conditions (see, e.g., FIG. 1). Examples of output data and insights include: i) apparent vascular permeability as a measure for transvascular diffusion due to CXCL12 stimulation, ii) hydraulic permeability of the vessel wall as a measure for transvascular convection in response to local intravascular and transvascular flow dynamics, iii) real-time detection of interstitial diffusion using cell-based sensing, and iv) hydraulic permeability as a measure of interstitial convection mediated by stromal fibroblast remodeling. Insights further include that endothelial cells integrate physical cues derived from matrix hyaluronic acid (HA) during angiogenesis induced by interstitial flow.

Hence, mimicking physiological mass transport and fluid mechanical forces is a technical problem in biomicrofluidic applications. Continuous perfusion of cell culture media ensures availability of nutrients and removal of waste products. However, existing methods for incorporating continuous and physiologically accurate fluid flows in microfluidic systems present many technical shortcomings. For example, programmable syringes, peristaltic pumps, and pressure pumps have been employed in an attempt to achieve precise and sustained flow rates in microfluidic devices. However, this setup involves careful consideration of tubing length and diameter used to connect the microfluidic device to the syringe pump. Moreover, the use of a syringe pump is also accompanied by operational challenges such as: 1) having expertise to prevent introduction of bubble(s) in the microfluidic device while connecting tubing, 2) presence of dead volume in the tubing, 3) relatively large space requirement and need for an external power supply to setup the pump within the context of limited incubator space, and 4) the limitation on the number of microfluidic devices that can be fluid powered using a single pump. Multichannel syringes, peristaltic pumps, and pressure pumps present similar issues. Often, syringe pumps can fluidically power only two microfluidic devices concurrently. To increase throughput beyond two microfluidic devices, a conventional approach is to either purchase multichannel syringe pumps having the capability to house 12 syringes or increase the number of syringe pumps in the lab inventory. Both of which are burdensome and costly which reduces accessibility. One may circumvent this issue by creating a hierarchy of tubing network with carefully selected diameters and lengths to ensure desired flow rate in the microfluidic devices. However, in this case, the flow rate in each microfluidic device is heavily influenced by whether the tubing is free of any surface fouling.

Typical systems and methods fail to provide a sustained and steady flow, can take up valuable incubator space, and the flow patterns are bidirectional and oscillatory which are uncharacteristic of unidirectional microphysiological flows. Moreover, the stepping motors of syringe pumps are subject to unsteady flow, especially when not properly lubricated. Some systems include a channel whose length can be modified to adjust the resistance to fluid flow. Existing approaches disadvantageously involve burdensome and costly acts such as creating different channel lengths to adjust the peripheral fluidic resistance and consideration of the channel geometry to achieve the desired flow rate.

The systems and methods described herein provide solutions to these and other technical problems described herein via a low-cost, easy-to-use, pumpless and tubeless miniaturized modular component to sustain hydrostatic pressure driven fluid flow in microfluidic devices for long-term applications (several hours to days). Notably, the proposed systems and methods do not rely on an external power source or expensive specialized lab equipment. Consequently, the experimental footprint is miniaturized compared to a microfluidic device controlled by a syringe pump, pressure pump, peristaltic pump, or a rocker platform. In some implementations, the systems and methods described herein can be realized in almost any laboratory. The varying combinations of hydrostatic pressure heads and the choice of the hydraulic resistor which might be a syringe filter in some implementations can facilitate a range of flow rates and physiological shear stresses. In some implementations, a custom or purpose-built component is provided (e.g., not based on a user trimming the syringe and/or affixing the filter). This custom component can be graduated in terms of flow rate for different specified heights to make it more ergonomic from a usability standpoint. Notably, the ability to generate differential flow rates also enables this component to be used for generating gradients.

In some implementations, the custom component can be graduated in terms of height (e.g., mm of H2O) and/or volume (e.g., ml of H2O). In some implementations, the custom component permits 12-24 hour intervals without user intervention to maintain flow rates within (+/−) a certain percent error (e.g., low <5%).

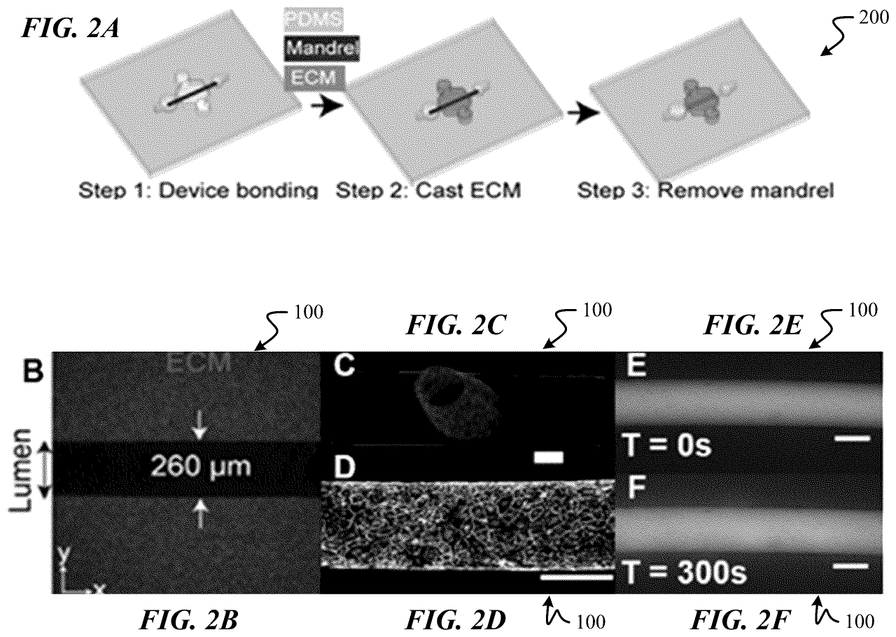

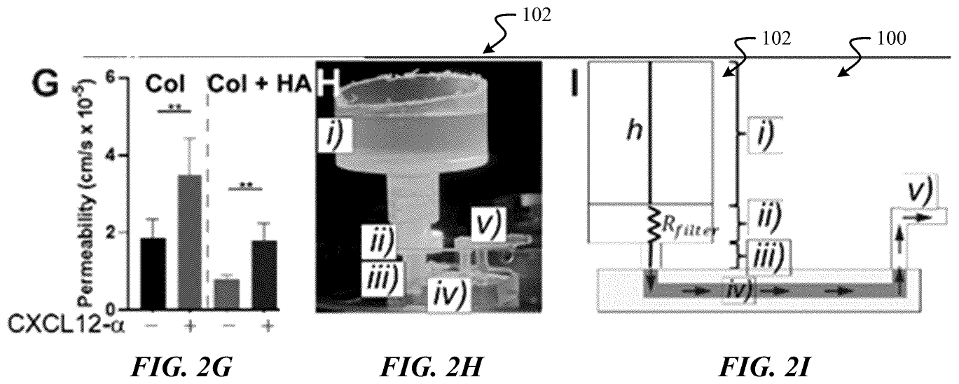

The pumpless perfusion setup will be incorporated with engineered microvessels. The setup for this configuration is shown in FIG. 2. Advantages of this perfusion setup are: 1) open top reservoir enables efficient media exchange via pipetting, 2) measurement of vessel permeability in situ (FIG. 2G), 3) intravascular pressure in the perfused microvessel can be readily adjusted via the hydrostatic pressure head of the media reservoir (FIG. 2H-I), and 4) a high resistance syringe filter positioned in series following the syringe reservoir (FIG. 2I) enables slow and continuous perfusion inside the downstream engineered microvessel without rapidly depleting media and the hydrostatic pressure head inside the reservoir. The magnitude of the hydrostatic pressure (P) is governed by the fluid height in the syringe:

$$P = ρ g Δ h \qquad [ 1 ]$$

where p is the density of the liquid, g is the acceleration of gravity, and Ah is the height difference of fluid in the syringe from the outlet. This hydrostatic pressure drives the flow and competes with the resistive force due to the syringe filter (determined experimentally) and the microfluidic channel (based on geometry of the channel). An electrical circuit analogy of this setup is shown in FIG. 2I. The flow rate is dictated by the hydrostatic pressure head and the equivalent resistance offered to the flow. In this case, the equivalent resistance is the sum of the syringe filter resistance and the microfluidic channel resistance. Typically, microfluidic channels have rectangular or circular cross-sections, and analytical solutions exist to compute the corresponding resistances. The syringe filter resistance depends on the filtration cross-section area (e.g. pore size and number of pores) and filter membrane thickness. Thus, by using filter membranes of different thicknesses, filtration area and pore size, a range of flow rates can be obtained for a specific microfluidic device at a fixed hydrostatic pressure head. Alternatively, for the same syringe filter and syringe combination, different flow rates can be obtained for a specific microfluidic device by using different initial hydrostatic pressure heads.

Based on experimentally obtained hydraulic resistance, we estimated that the initial flow rate for a hydrostatic pressure head of 40 mm and a syringe filter of 0.22 μm pore size and 4 mm filter diameter to be 6.7 μl/min with intravascular shear stress levels of 0.60 dyne/cm2. It is important to note that the flow rate obtained using this modular component decreases with time as the hydrostatic pressure head decreases. However, this issue can be mitigated by using a hopper with a larger diameter as well as resetting the hydrostatic pressure head at distinct time intervals (e.g., 12 or 24 hours).

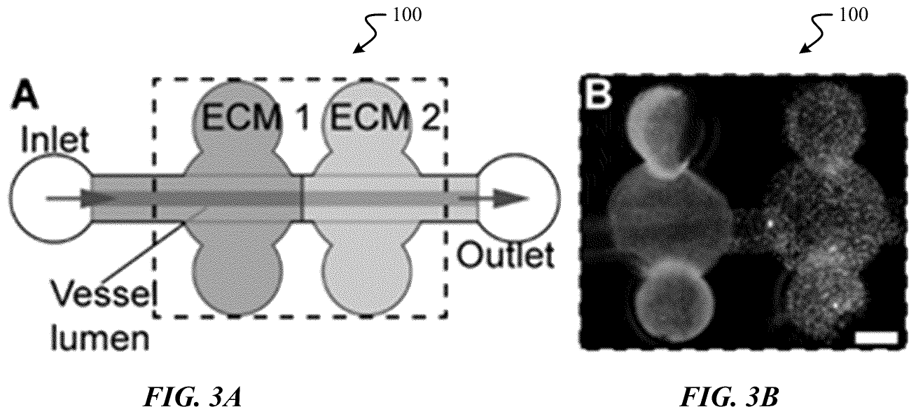

We also envision several enhancements to the pumpless microfluidic and modular microfluidic construct. One enhancement is spatially distinct ECM compartments along the same vessel segment (see, e.g., FIG. 3). This configuration will increase experimental throughput while also modeling heterogeneous tissue environments by specifying two different ECM compositions and/or restricting localization of fibroblasts in only one of the two adjacent ECM compartments. The second enhancement is systematically superimposing transvascular flow (TVF) across the blood vessel wall that becomes interstitial flow at distinct regions of the perfused microvessel

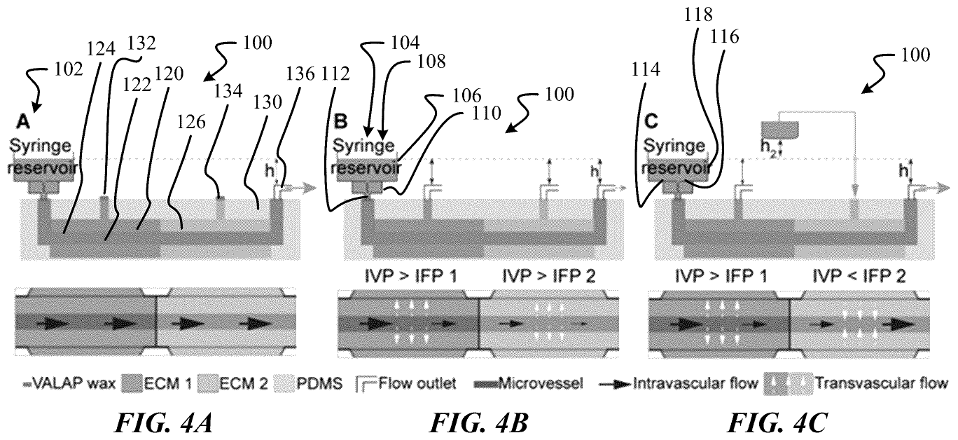

(FIG. 4). This configuration will model spatially heterogeneous fluid mechanics in tumor vasculature. We will achieve this capability through precise control of the transendothelial pressure difference between hydrostatic pressure in the luminal (IVP) and interstitial fluid pressure (IFP) domains. IVP will be the hydrostatic pressure head in the syringe reservoir (see, e.g., FIGS. 2H-2I) connected to the microvessel to enable pressure-driven intravascular perfusion (see, e.g., FIG. 4). To introduce outward TVF (or filtration), we will connect the ports for ECM 1 and ECM 2 (see, e.g., FIG. 4) to flow outlets, such that the interstitial fluid pressure of ECM 1 (IFP 1) and ECM 2 (IFP 2)=0. We will introduce low (1 μm/s) and high (10 μm/s) TVF. The TVF velocity will be determined numerically with a hydraulic resistance parameter that pairs in series the inverse of endothelial hydraulic conductivity (Lp in FIG. 1 ii) and interstitial hydraulic conductivity (K in FIG. 1). FIG. 4C depicts how we will introduce both outward and inward oriented TVF along the same microvessel. To introduce inward TVF (or reabsorption), the ports connected to ECM 2 (FIG. 3) will be connected to a fluid reservoir placed at a defined height above the syringe reservoir (h2 in FIG. 4C). We will determine the value for h2 with computational modeling, as previously described. Using local PIV, we can observe heterogeneous flow inside the microvessel, thereby capturing the effects of vessel leakiness of tumor vasculature.

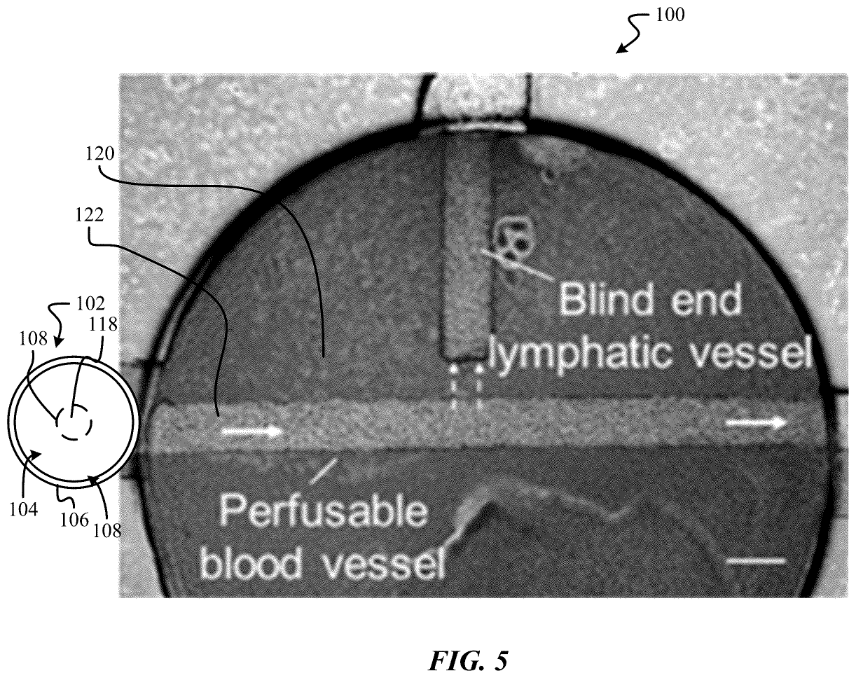

We can also use the pumpless perfusion setup to simulate pharmacokinetics and pharmacodynamics of intravenously administered drugs and recovery of fluid and molecules by lymphatics. FIG. 5 demonstrates our model for integrating the blood and lymphatic microvessels developed by our group into a single microtissue-engineered system (FIG. 5). This configuration will simulate drug transport processes in tissue between blood and lymphatic vessels. We can perfuse a selected drug through the blood vessel channel of the device, varying the concentration of infused drug in the open top pumpless perfusion setup to model pharmacokinetics of peak and trough levels measured in patients. This configuration also enables quantification of the kinetics of drug entry and distribution in the tissue using time-lapse fluorescence imaging. We can also use fluorescence microscopy to estimate the transvascular flux into the lymphatic vessel. This measurement will provide an estimate of the reduction of drug accumulation in tumor tissue due to drug clearance into the tumor draining lymphatic system. In the presence of interstitial flow, transvascular flux will occur primarily across the blind-ended region of the lymphatic vessel. We will thereby advance drug screening application using microfluidics to determine how tissue environments alter kinetics of drug delivery into our microtissue device.

FIG. 1 Transvascular and interstitial drug transport by either diffusion or convection. i): Molecules crossing the vessel wall by transvascular diffusion is a property of vascular permeability (P) and the concentration difference of the molecule between the vascular and interstitial space (Cv-Ci). ii) Bottom left: Transvascular convection of molecules is a property of the hydraulic conductivity of the vessel wall (Lp) and the difference between microvascular pressure and interstitial fluid pressure (Pv-Pi). Diffusion and convection also govern transport of molecules within the interstitial space. iii) Interstitial diffusion depends on the diffusion coefficient of a molecule (D) and the concentration gradient in the interstitial space (DCi). iv) Interstitial convection is a function of the hydraulic (or Darcy) permeability of the interstitial space (K) and the interstitial pressure gradient (DPi). Fluid in the interstitial space is absorbed by lymphatic vessels. In tumors, blood vessels are typically hyperpermeable, and lymphatic vessels are insufficient or nonfunctional. Consequently, the pressures inside and outside blood vessels are about the same (or Pv−Pi˜0), and interstitial pressure is relatively uniform (DPi˜0). Thus, both transvascular and interstitial convection of molecules are largely absent inside tumors.

FIG. 2. Micro-engineered and perfusable blood vessel. A) Fabrication steps of PDMS-based microfluidic device. This assembly permitted the specification of cell inlet and outlet ports and an ECM-derived hydrogel central region. B) Confocal microscopy image of fibrillar ECM in the microdevice and embedded with an open lumen structure ˜250-300 μm in diameter. C) Confocal projection of intact human umbilical vein endothelial cell (HUVEC) microvessel stained for VE-cadherin junctions (yellow) and DAPI for nuclei (blue). D) 3D confocal microscopy image of a vessel lumen stained for actin (phalloidin, red) and nuclei (DAPI, blue). E), F) Changes in fluorescence intensity of the tracer molecule (10 kDa FITC conjugated dextran) used to calculate apparent vascular permeability. Scale bars are 200 μm. G) Recombinant CXCL12-a (100 ng/ml) increased vessel permeability in Col only and Col+HA ECM. **, p<0.01. n=4 per condition. H) and I) open top and pumpless microfluidic control of perfusable microvessel (green in I). i) hydrostatic pressure head inside a trimmed 60 ml syringe: ΔP=ρgh where: ΔP—hydrostatic pressure, ρ—specific weight of water, g—gravity, h—fluid height difference between top of fluid filled reservoir and outlet; ii) syringe filer where resistance of the filter is a function of filtration area and filter membrane thickness: R_filter=f (filtration area, filter membrane thickness); iii) Luer lock connector in microdevice inlet; iv) engineered microvessel in the device; v) outlet. Arrows indicate the direction of fluid flow.

FIG. 3. Compartmentalized microvessel model. A) Distinct ECM regions along the same microvessel. B) Microvessel with two distinct ECM regions, embedded with different fluorescent beads Scale bar is 500 μm.

FIG. 4. Heterogeneous ECM regions and microvessel perfusion. A-C) depicts different microvessel perfusion states due to transmural coupling of intravascular, transvascular, and interstitial fluid flow. All conditions will be continuously perfused using the syringe reservoir described in FIG. 2. A) Ports for the ECM 1 and ECM 2 region are sealed with VALAP wax to block transvascular flow (TVF). B) Outward TVF becoming interstitial flow occurs in ECM 1 and ECM 2 due to equilibrated intravascular fluid pressure (IVP) being greater than the interstitial fluid pressure (IFP) in the ECM 1 and ECM 2 regions (IFP 1 and IFP 2 respectively). Smaller arrows for intravascular flow along the microvessel depict the expected reduction in perfusion due to outward TVF adjacent to the ECM 1 and ECM 2 regions. C) Outward TVF adjacent to ECM 1 (due to IVP>IFP 1) and inward TVF adjacent to ECM 2 (due to IVP<IFP 2). The sizes of the intravascular flow arrows depict the expected decrease in perfusion due to outward TVF (ECM 1) and increase in perfusion due to inward TVF (ECM 2).

FIG. 5. Integrated blood and lymphatic microvessel model. Image of engineered blood and lymphatic vessel within hydrogel of microtissue-engineered model. Solid arrow indicates intravascular perfusion. Dashed arrows indicate interstitial flow. Scale bar is 300 μm.

As shown in FIGS. 1-5, a system in the form of an automated liquid handling system 100 includes a liquid hopper 102 configured to contain a liquid within an internal volume 104. The liquid hopper 102 includes a hopper wall in the form of a hollow cylinder 106 defining an inlet 108. In some implementations, the hopper wall is not cylindrical. In some implementations, the hopper wall defines a custom geometric shape. The automated liquid handling system 100 includes a funnel 110 connected to the hollow cylinder 106 and defining an outlet 112 in fluid communication with the inlet 108. The automated liquid handling system 100 includes a hydraulic resistor mount in the form of a membrane mount 114 within the internal volume 104 and positioned on a sidewall 116 of the hollow cylinder 106. The automated liquid handling system 100 includes a hydraulic resistor in the form of a semipermeable membrane 118 spanning the internal volume 104 and configured to obstruct fluid flow between the inlet 108 and the outlet 112. The automated liquid handling system 100 includes a microfluidic device in the form of a synthetic microvessel 120 including an extracellular matrix (ECM) derived hydrogel, and defining a lumen 122 therethrough fluidly coupled to the outlet 112 of the fluid hopper 102. In some implementations, a positive hydrostatic pressure differential across the semipermeable membrane 118 is established based on a gravitational head of the liquid within the internal volume 104. When liquid flows from the outlet 112 and subsequently into the lumen 122, the liquid is permitted to perfuse at least one of: i) the lumen 122 and ii) a wall 124 of the synthetic microvessel 120.

In some implementations, the automated liquid handling system 100 includes a second synthetic microvessel 126 fluidly coupled to the synthetic microvessel 120. In some implementations, the second synthetic microvessel 126 includes a second ECM derived hydrogel of a different composition than the first ECM derived hydrogel. In some implementations, the automated liquid handling system 100 includes a microvessel container 130 housing the first synthetic microvessel 120 and the second synthetic microvessel 126, wherein the microvessel container 130 includes a first pressure port 132 associated with the first synthetic microvessel 120, and a second pressure port 134 associated with the second microvessel 126. In some implementations, the microvessel container 130 includes a lumen pressure port 136. In some implementations, the outlet 112 includes a needle hub. In some implementations, the outlet 112 includes a luer lock or a slip tip. In some implementations, when the liquid hopper 102 is coupled to the synthetic microvessel 120, the hollow cylinder 106 is situated gravitationally higher than the funnel 110 and the synthetic microvessel 120. In some implementations, the membrane mount 114 includes a shoulder between the hollow cylinder 106 and the funnel 110. In some implementations, the semipermeable membrane 116 is a disc or a wafer.

In some implementations, the automated liquid handling system 100 is an automated liquid handling system for high throughput. In some implementations, the system can be used in applications other than automated liquid handling. In some implementations, the system can be used for non-high throughput applications.

In some implementations, the system includes a controller having at least one processor and at least one non-transitory memory device storing instructions thereon that when processed by the processor cause the processor to obtain an input regarding the a channel geometry and shear stress; select, based on the input, a syringe filter value from a set of multiple values and select a hydrostatic pressure head value from a set of multiple values; and display, on a graphical user interface, one or more of the selected pressure head value and the syringe filter value.

As utilized herein with respect to numerical ranges, the terms “approximately,” “about,” “substantially,” and similar terms generally mean+/−10% of the disclosed values. When the terms “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as utilized herein to describe various implementations, are intended to indicate that such implementations are possible examples, representations, or illustrations of possible implementations (and such terms are not intended to connote that such implementations are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary implementations, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the automated liquid handling system 100 as shown in the various exemplary implementations is illustrative only. Additionally, any element disclosed in one implementation may be incorporated or utilized with any other implementation disclosed herein. Although only one example of an element from one implementation that can be incorporated or utilized in another implementation has been described above, it should be appreciated that other elements of the various implementations may be incorporated or utilized with any of the other implementations disclosed herein.

What is claimed is:

1. A system, comprising:
a liquid hopper including
a hopper wall defining an internal volume,
an inlet coupled to the hopper wall,
a funnel coupled to the hopper wall and defining an outlet in fluid communication with the inlet,
a hydraulic resistor mount coupled to the hopper wall between the inlet and the outlet, and
a hydraulic resistor coupled to the membrane mount; and
a microfluidic device comprising an extracellular matrix (ECM) derived hydrogel, and defining a lumen therethrough fluidly coupled to the outlet of the liquid hopper;
wherein when liquid flows from the outlet and subsequently into the lumen of the microfluidic device, the liquid is permitted to perfuse at least one of: i) the lumen and ii) a wall of the microfluidic device.

2. The system of claim 1, wherein the microfluidic device is a first microfluidic device and the ECM derived hydrogel is a first ECM derived hydrogel, and further comprising a second microfluidic device fluidly coupled to the first microfluidic device, wherein the second microfluidic device includes a second ECM derived hydrogel different than the first ECM derived hydrogel.

3. The system of claim 2, further comprising a microfluidic device container housing the first microfluidic device and the second microfluidic device,
wherein the microfluidic device container comprises a first pressure port associated with the first microfluidic device, and a second pressure port associated with the second microfluidic device.

4. The system of claim 1, wherein the microfluidic device container includes a lumen pressure port.

5. The system of claim 1, wherein the outlet includes a needle hub.

6. The system of claim 1, wherein the outlet includes a luer lock or a slip tip.

7. The system of claim 1, wherein when the liquid hopper is coupled to the microfluidic device, and
wherein the hopper wall is situated gravitationally higher than the funnel and the microfluidic device.

8. The system of claim 1, wherein the hydraulic resistor mount includes a shoulder between the hopper wall and the funnel.

9. The system of claim 1, wherein the liquid hopper is configured to contain a liquid, a polymer, a cellular suspension, a solid suspension, a soft solid suspension, a liquid solvent with solutes, nanoparticles, or DNA structures.

10. The system of claim 1, wherein the liquid hopper further includes a lid selectively coupled to the hopper wall and configured to reduce evaporation.

11. The system of claim 1, wherein the hopper wall is cylindrical.

12. The system of claim 11, wherein a diameter of the hopper wall is between 1 mm and 130 mm.

13. The system of claim 1, wherein the hydraulic resistor is a semipermeable membrane.

14. The system of claim 13, wherein the semipermeable membrane is a disc or a wafer.

15. The system of claim 1, wherein the hydraulic resistor includes a porous material or a microchannel.

16. The system of claim 1, wherein a hydraulic diameter of the hydraulic resistor is between 1 um and 500 um.

17. The system of claim 1, wherein a length of the hydraulic resistor is between 10 um and 100 mm.

18. The system of claim 1, wherein the hydraulic resistor defines a spiral shape.

19. The system of claim 1, wherein the microfluidic device includes a synthetic microvessel.

20. The system of claim 1, wherein a positive hydrostatic pressure differential across the hydraulic resistor is established based on a gravitational head of the liquid within the internal volume.
